(19) United States
(12) Patent Application Publication
(10) Pub. No.: US 2018/0049701 A1
(43) Pub. Date: Feb. 22, 2018

(54) MATTRESS FOR RESTING OR SLEEPING OF A PERSON

(71) Applicant: Emfit Oy, VAAJAKOSKI (FI)

(72) Inventor: Heikki Raisanen, Muurame (FI)

(21) Appl. No.: 15/557,153

(22) PCT Filed: Mar. 14, 2016
(86) PCT No.: PCT/FI2016/050157

§ 371 Date: Sep. 11, 2017

(30) Foreign Application Priority Data
Mar. 13, 2015 (FI) 20155170

(51) Int. Cl. A61B 5/00; A61B 5/024; A61G 7/05; A61B 5/08; A61B 5/113

(52) U.S. Cl. CPC A61B 5/6892; A61B 5/02444; A61B 5/02405; A61B 5/1102; A61B 5/0816; A61B 5/4806; A61B 5/113; A61G 7/05

(57) ABSTRACT

A mattress for resting or sleeping of a person includes a sensor arranged in connection with the mattress for determining the heart rate and/or the heart rate variation and/or breathing disturbances and/or the respiratory frequency and/or the categories of sleep that are affected by the person's movements and/or micromovements. An electronic unit is connected to the sensor with a cable. Canals are arranged within the mattress for the sensor, the electronic unit and the cable. In addition, the sensor is placed near a first end of the mattress while the electronic unit is located at the other end of the mattress.

The invention is related to a mattress for resting or sleeping of a person with a sensor arranged in connection therewith for determining the heart rate and/or the heart rate variation and/or breathing disturbances and/or the respiratory frequency and/or the categories of sleep that are affected by the person's movements and/or micromovements and wherein an electronic unit is connected to the sensor with a cable.

Sensors installed in a bed in connection with a mattress, above or below it, for determining breathing, heart rate, heart rate variation and movement activity based on micromovements of the human body are known. It is also known that this information can be further utilised, for example, for determining the categories of sleep of a sleeping person (REM, deep sleep and light sleep). Sensors also exist, the raw signal produced by which can be used to diagnose, by a visual examination, whether a person has sleep apnea or even cardiac arrhythmias. Likewise, the use of such sensors for monitoring vital functions of a patient, as well as for monitoring lying on a bed and particularly leaving the bed of elderly people is known.

It is also known that one or more sensors can be as if integrated into a bed sheet that is pulled over a mattress. Moreover, it is known that a sensor may be thin, composed of several plastic membranes, based on a piezoelectric membrane, such as a PVDF membrane, or a ferroelectric membrane, such as a polypropylene membrane containing electrostatically charged flat gas bubbles. Furthermore, thicker and rather rigid sensors including piezoceramic crystals are known. Other techniques are also used. Prior art includes, for example, the so-called FSR (force-sensing-resistor), as well as a system in so-called smart beds where a sensor in an air mattress is used to measure micromovements caused by small variations in the air pressure.

The object of the invention is to provide a novel mattress that enables placement of a sensor and the related signal processing device in a bed, associated with the mattress of the bed, more unnoticeably and easily than before. The characteristic features of this invention become apparent from the appended claims. An adaptation is arranged within the mattress for the sensor assembly for providing a so-called smart mattress. For example, when a system that monitors the vital functions and therethrough the wellbeing and long-term trends and resting is to be placed in a bed of a person suffering from dementia, it is challenging to do this so unnoticeably that the person him- or herself does not notice it, because such people typically would like to remove or even break such objects. Often, users also look for solutions as unnoticeable as possible for aesthetic reasons, as well as solutions that are not in the way when changing bed linen. Another object of the invention is to enable replacement of the electronic unit that contains a damaged or technologically outdated sensor and a signal processing device inside the mattress without the need of replacing the entire mattress because of this. Particularly, in hospital and elderly home environments, good hygiene is important and therefore, an object of the invention is to provide a mattress where the sensor system and electronics are completely within the mattress without the need of arranging any holes for their introduction.

The invention is described below in detail by referring to the appended drawings, which illustrate an embodiment of the invention, in which:

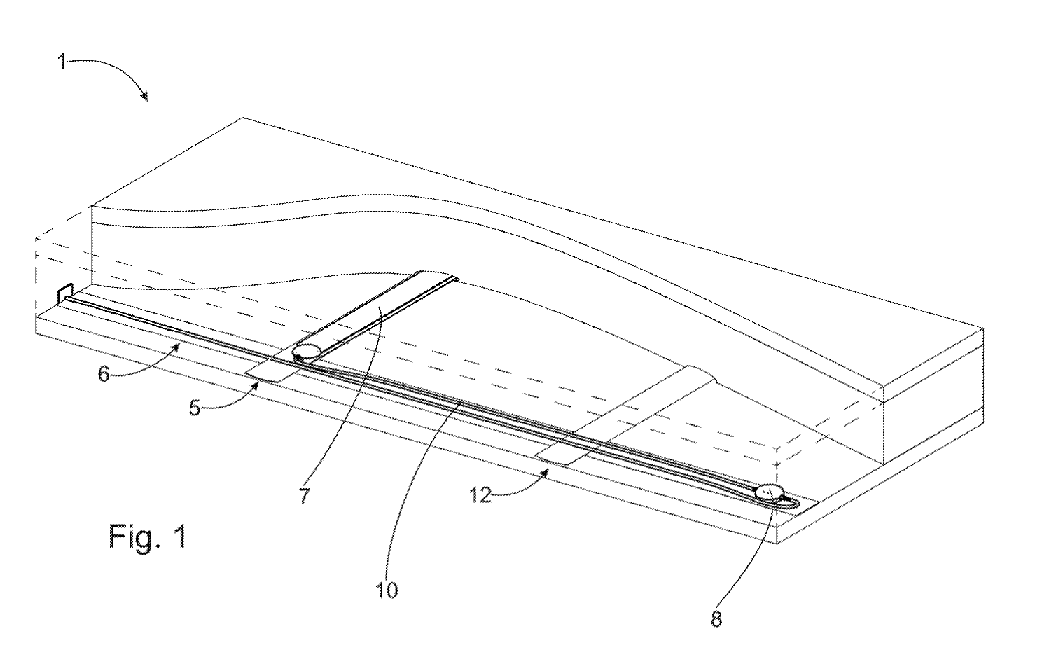

FIG. 1 shows a cross-sectional view of a mattress according to the invention,

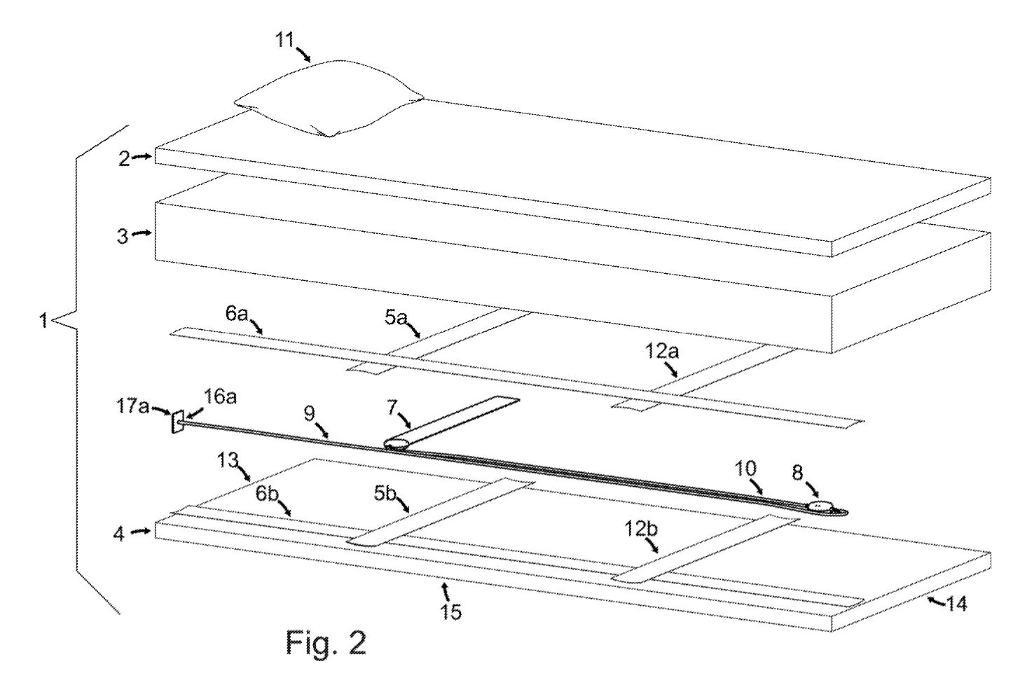

FIG. 2 shows an exploded view of a mattress according to the invention,

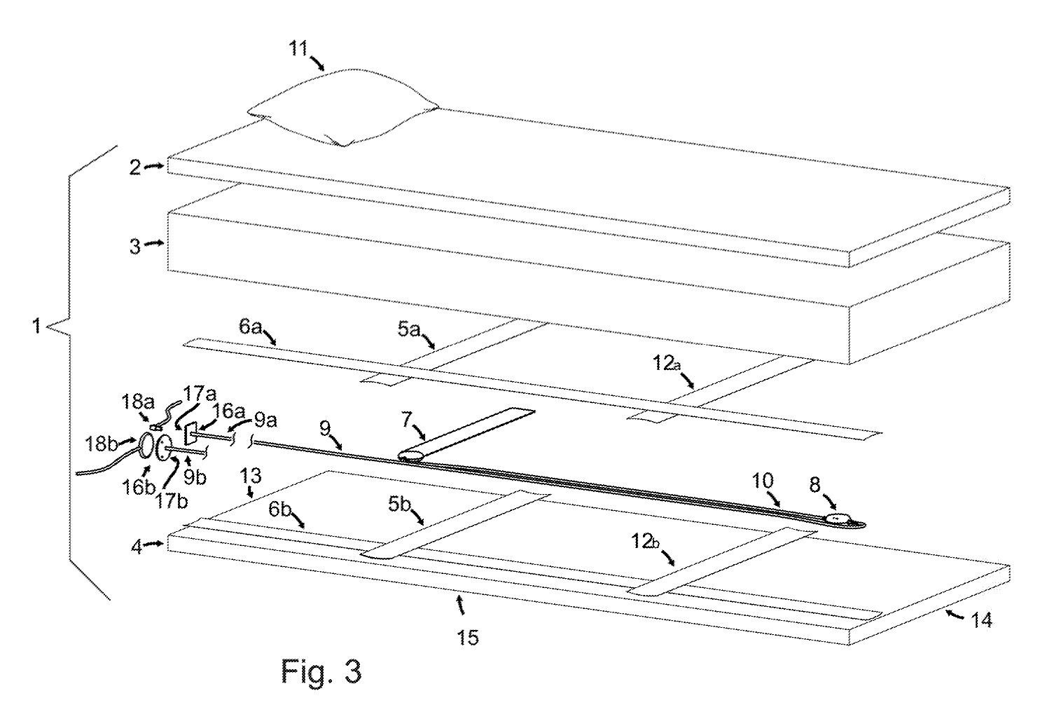

FIG. 3 shows a modification of a mattress according to the invention.

FIGS. 1, 2 and 3 illustrate a traditional mattress 1 made of foamed plastic. Generally, the mattress 1 is arranged to be composed, in the thickness direction, of two, three or more layers 2, 3 and 4 with different rigidities. Mattresses may also have different rigidities in the longitudinal direction to correspond to different surface pressures applied to the mattress by different parts of the human body, such as shoulders and hips. For purposes of simplification, FIGS. 2 and 3 display a three-layer structure where, however, the rigidities are equal in the longitudinal direction of the entire mattress 1.

In this embodiment of the invention, when gluing together the three different layers of the mattress during its manufacture, two canals 5 and 6 have been placed, according to the invention, between two of its different opposing layers. The layers are not always necessarily glued together, particularly, if the entire mattress is encircled by a baglike cover protecting from microbes and body fluids; this cover can also be completely hermetic. The canal 5 is set crosswise relative to the lengthwise direction of the mattress 1 to enable installation of a sensor 7 therein at a later stage. An electronic unit 8 is connected to the sensor 7 with a cable 10. Thus, in an advantageous embodiment of the invention, canals 5, 6, 12 have been arranged within the mattress 1 for the sensor 7, the electronic unit 8 and the cable 10. In addition, the sensor 7 is placed near the first end 13 of the mattress 1 while the electronic unit 8 is located at the other end 14 of the mattress 1. The electronic unit 8 is meant for the processing of the sensor signal and further, for sending it via the Internet, for example, to a server, via which the signal and/or data computed from it can be viewed, among other things. The electronic unit 8 can be placed within the mattress 1 at the end located the furthest from the canal 5, for a reason explained later in this text.

Advantageously, the electronic unit 8 includes a connection cable 9 that extends to the end 13 opposing that end 14 of the mattress 1 which is situated on the side of the electronic unit 8. The canal 6 is disposed lengthwise to the mattress 1 and perpendicular to the canal 5 to enable conveyance of at least the sensor connection cable 9 within the mattress 1.

In a glued mattress construction, the canal 5 is advantageously composed of thin plastic membranes made of PVC, PP or other plastic, for example, and cut at suitable widths and lengths, such as in a thickness class of 0.5-1.0 mm, however, these may also be made of another material, such as thin metal, in the case of a spring mattress referred to later. Canals may also be provided as a mere shape, such as a chamfer, on the surface of the layer. It is essential that the space arranged within the mattress for the sensor assembly is composed of a minimum of two canals 5 and 6, advantageously set perpendicular to each other. In turn, canals are advantageously composed of two superimposed plastic membranes, referred to above, advantageously set against each other and with sliding surfaces that are not glued together, excepting at their very edges, if needed. The reference numbers of the parts forming the canal 5 are 5a and 5b. The same numbering system is used for canals 6 and 12.

The canal 5 set crosswise to the mattress 1 is advantageously arranged approximately in such a way that when a person is resting or sleeping on the bed, the canal is approximately at the height of the person's heart when viewing along the direction of length of the mattress, so that the sensor can well pick up both the heartbeat recoil and the lung breathing movement. To enable installation of the mattress in either direction, the number of these transverse canals must be two with both of the canals 5 and 12 approximately at an equal distance from the ends 13 and 14 of the mattress. More generally, the canals 5 and 6 are arranged at an equal distance from the end 13, 14 located on the canal side of the mattress 1. This facilitates installation when the mattress is already in use in a person's bed and also enables turning the mattress 180 degrees at times during its yearslong use, in addition to the possibility of only turning it upside down. The transverse canal 5 extends up to the outer edge 15 of the mattress at least on the other side. It is advantageous but not necessary that it extends to the outer edge on both sides, since installation can then be easier when the mattress is already in use.

The transverse canal 5 is internally connected to a canal 6 set in the lengthwise direction of the mattress 1. Again, the canal 6 extends at least to the end to which the transverse canal is closer. More precisely, the canal 5 is closer to the end 13 and the canal 12 is closer to the foot end 14. It is advantageous that the canal 6 also extends at least almost or completely to the other end, i.e. to the foot end 14 of the mattress 1, due to a reason related to the electronic unit, which will be explained later. It is the most advantageous that the canal 6 extends completely from end to end in the lengthwise direction of the mattress 1, because this enables turning the mattress 180 degrees at a later stage. The canal 6 set in the lengthwise direction of the mattress is arranged in such a way that it is near the edge 15 of the mattress 1, however, at such a distance that the layers 2, 3 and 4 of the mattress 1 can be glued together at the long edge, too, if desired. Generally, the mattress 1 has a minimum of two layers 2, 3, 4, between which the canals 5, 6, 12 are arranged.

The width of the transverse canal is adapted suitable for the sensor to be installed in it. For example, the canal could be 80 mm wide in the internal area where its top and bottom side are not glued together. In this case, a sensor with its width in the class of 60 mm, such as a PVDF sensor or a ferroelectric sensor, which are known to be very thin and membrane-like, can be easily inserted to it. The canal may also be wider or narrower than this. The width of a lengthwise-running canal or canals, if these are arranged to run close to both long sides, is such that the cable connecting the sensor and the electronic unit can be easily threaded to it at a later stage; this cable can also be a flat cable realised by a compression technique and advantageously shielded.

When the canals are arranged as described above, the sensor complete with its cables can be installed within the mattress at a suitable later stage. This advantageously also includes the related electronic unit complete with its wireless transmitter. Since a mattress is typically used for as long as 10 years or even more, it is possible that the electronic unit 8 or the sensor 7 or the connection cables 9 or the cable 10 become damaged during use and therefore, it is particularly important that they can be replaced without the need of replacing the mattress itself. It is also possible that the technology advances and better performing solutions become available, in which case it may also be necessary to have the possibility to replace the sensor and the electronic unit. Cables can also be called leads.

For example, the installation can be performed by first inserting the sensor 7 to the desired transverse canal 5 or 12. Thereafter, a rigid pulling tool, such as rigid wire, is inserted from the end of the mattress 1 to its lengthwise-running canal. The tool helps to pull the sensor cable 10 to run up to the end of the mattress 1 through its canal 6. The connection cable 9 of the sensor 7 may end at a female connector 16, such as a typical 6.35 mm stereo jack, which is adapted, for example, to a plastic plate 17 that remains inside the cover of the mattress 1. Alternative end caps 9a and 9b for the connection cable are shown in FIG. 3. The cover is typically made of fabric or plastic and closed with a zipper. A place for the plastic plate 17a is arranged in the end fabric or plastic where it stays well in place while only the lead hole is visible inside the mattress. Furthermore, the signal processing device or the electronic unit can be taken further with another cable having a male connector arranged in an angle of 90 degrees, such as a 6.35 mm stereo plug 18a, if the female connector is a stereo jack 16a. In this way, the electronic unit, which typically has a transceiver that generates electromagnetic radiation, such as WiFi or Bluetooth or mobile data, will not remain near the head of the person sleeping in the bed but can be conveniently placed on the floor at a greater distance from the sleeping person. Generally, the electronic unit 8 has a wireless transceiver.

However, due to aesthetic and hygienic reasons and the requirement of unnoticeability, it is often desired that the electronic unit is also arranged within the mattress. In the solution according to the invention, the sensor cable 10 can be conveyed along the lengthwise-running canal 6 to the foot end 14 and the electronic unit 8 can be placed inside the canal so that it will be, together with its transceiver, at a sufficient distance from the body, and particularly the head, of a sleeping person. The power cord, more generally, the connection cable 9 for the electronic unit 8 can be conveyed along the canal 6 from the end 13 on the side of the pillow 11 and a female connector 16a can be arranged for it in the same way as described above, on the plastic plate 17a adapted within the mattress cover.

Power for the electronic unit can also be conducted inductively. In other words, the electronic unit 8 is provided or associated with an inductive current input, in which case the mattress can be handled, such as vacuumed, washed or dampwiped and disinfected, as well as fitted with bed linen without disturbing loose cables. On the bottom of the bed, the power radiator of the inductive method can be adapted in the position where the electronic unit 8 and the inductive power receiver in the mattress, fixedly or separately adapted in connection therewith, is arranged to be set. Due to the simple representation of the figures, the inductive receiver 16b is shown in the vicinity of the power cord 9b, adapted to the plate 17b, and the power transmitter 18b is illustrated so as to be fixed in a suitable place on the headboard of the bed. However, because of electromagnetic radiation, its position would generally be adapted against the bottom of the bed, suitably at the foot end 14, at a greater distance from the body. Generally, an inductive current receiver 16b is connected to the electronic unit 8. In FIG. 3, the receiver 16b is arranged at the end 13 of the mattress 1, but it can also be placed at the foot end 14 or on the underside of the mattress 1. Thus, depending on the location of the receiver 16b, the current transmitter 18b is placed in the corresponding position on the headboard or the bed bottom.

Electromagnetic radiation can also be prevented by switching off the wireless transceiver for the time that the person is in the bed, in which case its electronic unit can be placed completely attached to the sensor. In this case, however, a quick bed exit alarm will be lost in the monitoring application, which is important when monitoring aged patients with dementia.

In addition to foam mattresses, spring mattresses are very common. In these, the canals required by sensor and cable assemblies may be arranged within the mattress in such a way that, at the canals, springs are cut approximately in the middle in the longitudinal direction, the canals thus being disposed between the springs. However, in order to stay in place, they can be fastened to adjacent springs, for example.

Many types of foam mattresses exist, for example, so-called memory-foam mattresses. Mattresses that are mainly made of natural materials, such as so-called futon mattresses, also exist, which, however, greatly resemble foam mattresses, and canals can be arranged within these in the same way as in a mattress.

Correspondingly, in an air mattress bed, it has been possible to divide the height of the air cushions provided in the thickness direction of the mattress into two opposing cushions at the canals so that it has been possible to correspondingly arrange the canals between these and hence, within the mattress.

The canals are not necessarily exactly perpendicular to each other, but particularly the lengthwise-running canal of the mattress can basically also start from one corner of the short side and end in the other corner on the opposite side.

Advantageously, the mattress is provided with a bag or cover that prevents access of microbes and fluids to the mattress; this bag or cover can be even hermetic. In this case, the sensor and the electronic unit, as well as the related cables are well protected. High hygienic requirements can be reliably fulfilled when using an inductive power supply and a wireless data transceiver, such as WiFi, Bluetooth, Zigbee, mobile data or another similar technique.

It is apparent for one skilled in the art that the different embodiments of the invention can be manifold and vary within the claims set forth below.

1-15. (canceled)

16. Mattress for resting or sleeping of a person with a sensor arranged in connection therewith for determining the heart rate and/or the heart rate variation and/or breathing disturbances and/or the respiratory frequency and/or the categories of sleep that are affected by the person's movements and/or micromovements, and the mattress has a minimum of two layers, between which there is an electronic unit connected to the sensor with a cable, and between two layers there are canals composed of opposing parts which are plastic membranes set superimposed against each other with sliding surfaces, and
the said canals are arranged within the mattress for the sensor, the electronic unit and the cable, wherein one canal is lengthwise-running and the other canal is transverse extending up to the outer edge of the mattress at least on the other side, and the said canals are mainly arranged perpendicular to each other and connected together for allowing inserting and replacing of the sensor and the cable connected thereto, and the sensor is arranged near the first end of the mattress while the electronic unit is located at the other end of the mattress.

17. Mattress according to claim 16, characterised in that power for the electronic unit is arranged inductively.

18. Mattress according to claim 16, characterised in that the electronic unit has a wireless transceiver.

19. Mattress according to claim 16, characterised in that the electronic unit includes a connection cable extending to the end that opposes that end of the mattress which is situated on the side of the electronic unit.

20. Mattress according to claim 16, characterised in that the opposing parts are connected together at opposite edges.

21. Mattress according to claim 16, characterised in that the mattress includes another transverse canal.

22. Mattress according to claim 21, characterised in that the canals are arranged at an equal distance from the end of the mattress on the canal side.

23. Mattress according to claim 21, characterised in that at least one of the canals extends at least to one long side of the mattress, to its vertical surface, approximately in the area where the chest of the sleeping person will be located when viewing in the lengthwise direction of the mattress.

24. Mattress according to claim 16, characterised in that the lengthwise-running canal of the mattress extends at least to one end of the mattress.

25. Mattress according to claim 24, characterised in that the lengthwise-running canal is arranged at the edge of the mattress.

26. Mattress according to claim 16, characterised in that the mattress has a cover for protecting against microbes and fluids.

27. Mattress according to claim 17, characterised in that an inductive current receiver is connected to the electronic unit.

28. Mattress according to claim 27, characterised in that the receiver is arranged at the end of the mattress or on the bottom of the mattress.
